(19) United States
(12) Patent Application Publication
(10) Pub. No.: US 2017/0281289 A1
(43) Pub. Date: Oct. 5, 2017

(54) FEMORAL DRAPE

(71) Applicants: Ezad Ahmad, Dalton, GA (US); Patrick Haden Wilder, Chatsworth, GA (US)

(72) Inventors: Ezad Ahmad, Dalton, GA (US); Patrick Haden Wilder, Chatsworth, GA (US)

(21) Appl. No.: 15/474,576

(22) Filed: Mar. 30, 2017

Related U.S. Application Data
(60) Provisional application No. 62/315,056, filed on Mar. 30, 2016.

(51) Int. Cl. A61B 46/27; A61B 46/00

(52) U.S. Cl. CPC A61B 46/27; A61B 46/40; A61B 2046/205

(57) ABSTRACT

A sterile femoral drape intended to be placed over a surgical patient lying on an operating table to isolate the patient during the surgical procedure, the drape comprising a top sheet of bio-impermeable material of a length and width greater than a conventional operating table such that the top sheet fully encompasses the patient and drapes over the lateral side edges and the bottom (foot) edge of the table. Femoral access holes located proximate to the patient's groin are provided to allow access to the patient's femoral artery during heart catheterizations or other surgical procedures. The drape comprises left and right arm pockets formed underneath the bio-impermeable sheet into which the patient's respective left and right arms are inserted. The patient's arms being positioned within and supported within the arm pockets, fully isolates the patient's arms and hands from the femoral access site, thereby assuring that the patient's hands do not contaminate the femoral access site.

### CROSS-REFERENCE TO RELATED APPLICATIONS

This application claims the benefit of pending provisional application No. 62/315,056, filed Mar. 30, 2016.

### BACKGROUND OF THE INVENTION

### Field of the Invention

This invention relates to surgical drapes. More particularly, this invention relates to surgical drapes having an access to a patient's femoral artery.

### Description of the Background Art

Presently, there are many forms of surgical drapes used in operating rooms to isolate the patient lying on the operating table from contaminating the operating site. Representative surgical drapes are set forth in the following patents and patent applications: U.S. Pat. Nos. 3,799,161, 4,119,093, 5,140,996, 5,640,975, 6,199,553, 6,298,855, 6,843,252, 7,114,500, 7,958,894, 8,079,365, 8,100,130, 8,322,345, 8,371,306, 8,863,747, 2008/0283064, 2012/0298115, 2013/0125901, the disclosures of which are each hereby incorporated by reference herein.

As shown representatively in U.S. Pat. No. 8,863,747, prior art drapes may include one or more openings for convenient access to the patient's femoral artery. Typically, the femoral access opening comprises an adhesive about its inner periphery to adhere to the patient's skin about the surgical site with the intended purpose of preventing contamination from the other areas of the patient's body to the surgical site. Unfortunately, however, often times the patient's hands may inadvertently move underneath the drape to the access opening and exert enough force to peel off the adhesive from the patient's skin, thereby contaminating the femoral surgical site. This problem is particularly acute in regard to femoral artery surgical sites due to the proximity of the patient's hands to the femoral surgical area.

Therefore, it is an object of this invention to provide an improvement which overcomes the aforementioned inadequacies of the prior art devices and provides an improvement which is a significant contribution to the advancement of the surgical drape art.

Another object of this invention is to provide a femoral drape that includes means for isolating the patient's hands from the femoral artery surgical site to assure that the patient's hands do not inadvertently peel off or dislodge the adhesive that adheres the femoral drape about the access opening.

Another object to this invention is to provide a femoral drape that includes a means for supporting the patient's arms alongside the patient's body and thereby obviate the need for arm boards.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a sterile femoral drape intended to be placed over a surgical patient lying on an operating table to isolate the patient during the surgical procedure. The drape comprises a top sheet of bio-impermeable material of a length and width greater than a conventional operating table such that the top sheet fully encompasses the patient and drapes over the lateral side edges and the bottom (foot) edge of the table. Access holes located proximate to the patient's groin are provided to allow access to the patient's femoral artery during heart catheterizations or other surgical procedures. The access holes may optionally include an adhesive around the inner periphery thereof to removably adhere the inner periphery to the patient's skin about the intended femoral access site, thereby isolating the femoral access site from the areas underneath the patient.

The drape of the present invention further comprises left and right arm pockets formed underneath the bio-impermeable sheet into which the patient's respective left and right arms are inserted. The arm pockets each comprise another sterile sheet of bio-impermeable material. The lateral and bottom edges of the arm pocket sheets are hermetically sewn to the underside of the top sheet. The upper edge of the arm pocket sheets remains open allowing the patient's arms to be inserted into the respective arm pocket and positioned between the top sheet and the respective arm pocket sheet.

The width of each the arm pocket sheet is sufficiently wide to allow the patient's arm to be easily inserted therein. The arm pocket sheets are separated from one another to the approximate width of a human torso so that they are positioned at the lateral edges of a conventional operating table, thereby supporting the patient's arms along the patient's sides and optionally obviating the need for arm boards to support the patient's arms during surgery. Notably, that the patient's arms are positioned within and supported within the arm pocket, fully isolates the patient's arms and hands from the femoral access site, thereby assuring that the patient's hands do not contaminate the femoral access site as might otherwise occur in prior art femoral drapes in the event the patient's fingers push up on the underside of the top sheet and inadvertently peel off or dislodge the adhesive seal formed between the periphery of the access hole and the patient's skin about femoral access site.

The femoral drape of the invention provides a total separation of patient and the surgical site because of the pockets that are formed on the underside of the drape that are designed to both support the patient's upper extremities and create a barrier from the surgical site. The lack of access to the surgical site by the patient minimizes the need for adhesive at the surgical site, thus creating less damage and discomfort when removing the drape. In addition, the pockets provide IV access windows and full support of the patient's upper extremities, thus eliminating the need for arm boards. It also substantially reduces the risk of a patient's hand being caught in the table during a procedure.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a plan view of femoral drape of the invention draped over a typical patient laying on a surgical table; and

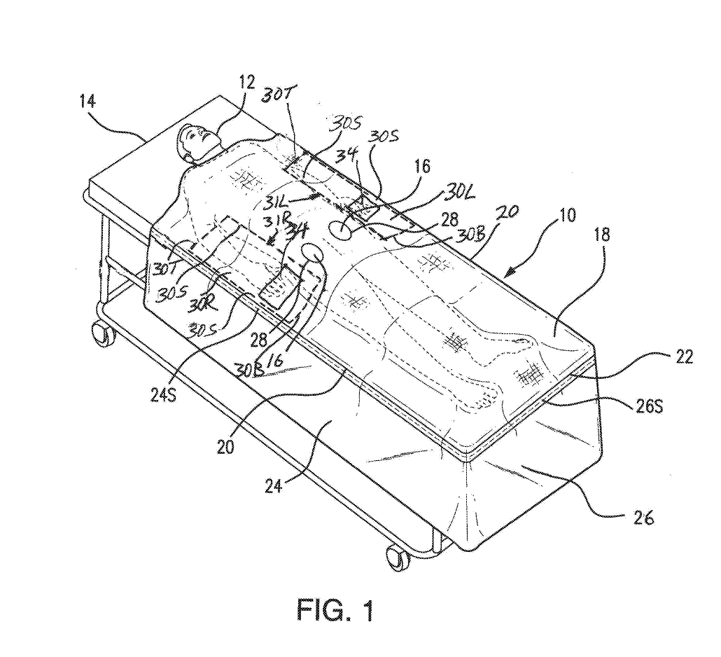

FIG. 2 is a plan view of the underside of the femoral drape of the invention showing the relative configuration and placement of the arm pocket sheets sewn to the underside of the top sheet.

Similar reference characters refer to similar parts throughout the several views of the drawings.

### DETAILED DESCRIPTION OF THE PREFERRED EMBODIMENT

The femoral drape 10 of the invention comprises a generally rectangular sheet of bio-impermeable material that is intended to be draped over the body 12 of a patient lying on an operating table 14 to minimize cross contamination between the patient's body 12 and femoral operating site(s) 16.

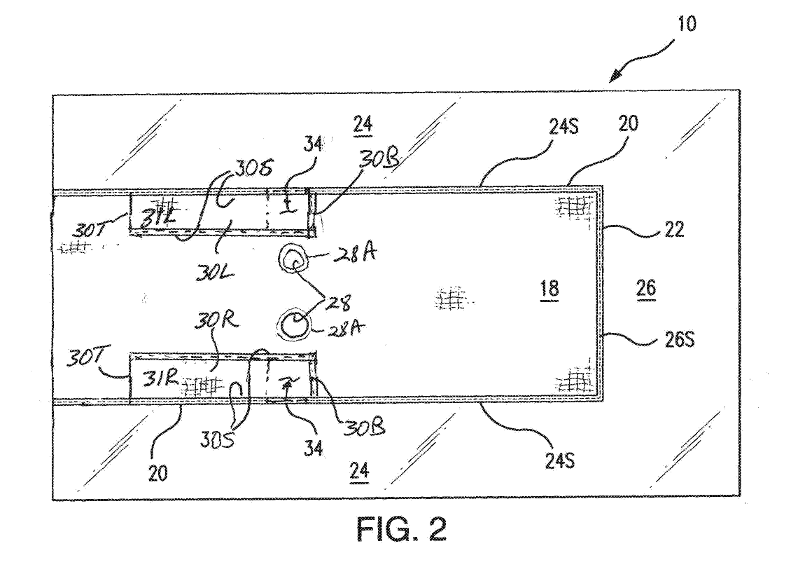

The femoral drape 10 comprises a top sheet 18 greater in length than the patient's body and wider than the width of the operating table 14 so as to drape over the lateral edges 20 of the table 14 and the bottom or foot edge 22 of the table 14. The femoral drape 10 may additionally optionally include side extensions 24 and bottom extension 26 sewn about the periphery of the lateral edges 20 and bottom edge 22, respectively of the top sheet 18 at seams 24S and 26S. Preferably, the top sheet comprises a generally opaque, bio impermeable material whereas the extensions 24 and 26 comprise a transparent or translucent, bio impermeable material. The femoral drape 10 further comprises femoral access openings 28 located proximately to the femoral operating site(s) 16 of the femoral artery of the patient's body 12 allowing convenient access for cardiac catheterizations or other surgical procedures. An adhesive layer 28A is optionally applied to the periphery of the access openings 28 (see FIG. 2) to removably adhere to the patient' skin about the periphery of the femoral operating site(s) 16 and isolate the same.

Left and right sheets of bio impermeable material 30L and 30R are positioned underneath the top sheet 18 to form respective left and right arm pockets 31L and 31R. The arm pocket sheets 30L and 30R each comprises a generally rectangular configuration having a width sufficiently wide and long to allow a patient's arm to be easily inserted therein. The left and right lateral side edges 30S and the bottom edge 30B each pocket sheet 30L and 30R are fastened to the underside of the top sheet 18 such as by sewing. The top edge 30T of each of the pocket sheets 30L and 30R is left opened (i.e., not fastened).

It is noted that the top edge 30T of each of the pocket sheets 30L and 30R is positioned appreciably downward along the length of the top sheet 18 to an area approximately below the patient's armpits thereby allowing the patient's arm 12A to be inserted into the respective arm pockets 31L and 31R while allowing the top edge of the top sheet 18 to remain about the patient's neck area.

An access opening 34 in the top sheet 18 into the respective arm pockets 31L and 31R may be provided approximate to each of the patient's hands to allow access to the patient's hands without having to reach under the drape 10 (e.g., for connecting IV lines, pulse oximeters, and the like).

Immediately prior to surgery, the patient's arms 12A may be inserted between the respective arm pockets 31L and 31R. The arms 12A are therefore supported along the lower sewn edges 30S of the respective arm pockets 31L and 31R, thereby preventing the patient's arms 12A from otherwise falling off of the operating table 14. Moreover, arm boards (not shown) may be eliminated altogether.

An important feature of the present invention is the arm pockets 31L and 31R which isolate patient's arms 12A from the femoral operating site 16. In this manner, the patient's arms, particularly the patient's hands, are prevented from working down toward the femoral operating site 16 and contaminating the same. Moreover, it is noted that even when adhesive 28A is employed around the femoral access openings 28, the arm pockets 31L and 31R prevent the patient's hands from inadvertently or unintentionally working the adhesive 28A around the access opening 28 off of the patient's skin and contaminating the operating site 16.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A femoral drape comprising, in combination:
a top sheet that is intended to be draped over the body of a patient lying on an operating table, said top sheet being greater in length than the patient's body and wider than the width of the operating table so as to drape over the lateral edges of the table and the foot edge of the table;
said top sheet comprising at least one femoral access opening located proximately to a femoral operating site of the femoral artery of the patient's body, thereby allowing convenient access for cardiac catheterizations or other surgical procedures; and
left and right arm pocket sheets positioned along said top sheet to form respective left and right arm pockets.

2. The femoral drape as set forth in claim 1, wherein said left and right sheets of bio impermeable material are positioned underneath said top sheet.

3. The femoral drape as set forth in claim 1, further including side extensions and bottom extension fastened about the periphery of respective lateral edges and bottom edge of said top sheet.

4. The femoral drape as set forth in claim 1, wherein said top sheet comprises a generally opaque, bio impermeable material.

5. The femoral drape as set forth in claim 3, wherein said side extension and said bottom extension comprise a transparent or translucent, bio impermeable material.

6. The femoral drape as set forth in claim 1, wherein an adhesive layer is applied to the periphery of said access opening to removably adhere to the patient' skin about the periphery of the femoral operating site and isolate the same.

7. The femoral drape as set forth in claim 1, wherein said arm pocket sheets each comprises a generally rectangular configuration having a width sufficiently wide and long to allow a patient's arm to be inserted therein.

8. The femoral drape as set forth in claim 7, wherein left and right lateral side edges and a bottom edge each said pocket sheet are fastened to an underside of said top sheet with a top edge of each of said pocket sheets being left opened.

9. The femoral drape as set forth in claim 1, wherein said top edge of each of said pocket sheets is positioned appreciably downward along said length of said top sheet to an area approximately below the patient's armpits thereby allowing the patient's arm to be inserted into said respective arm pockets while allowing said top edge of said top sheet to remain about the patient's neck area.

10. The femoral drape as set forth in claim 1, further including an access opening in said top sheet into respective said arm pockets approximate to each of the patient's hands to allow access to the patient's hands.

11. The femoral drape as set forth in claim 1, wherein the patient's arms while inserted between respective said arm pockets are supported along of respective said arm pockets, thereby preventing the patient's arms from otherwise falling off of the operating table.

12. The femoral drape as set forth in claim 1, wherein said arm pockets isolate the patient's arms from the femoral operating site thereby the patient's hands are prevented from contaminating the same.

13. The femoral drape as set forth in claim 1, wherein said top sheet comprises a bio impermeable material.

14. The femoral drape as set forth in claim 1, wherein said arm pocket sheets comprise a bio impermeable material.
